(19) United States
(12) Patent Application Publication
(10) Pub. No.: US 2015/0142324 A1
(43) Pub. Date: May 21, 2015

(54) Healthbeat of plants

(71) Applicants: Shuza Binzaid, San Antonio, TX (US); Shaneel Shuza, San Antonio, TX (US)

(72) Inventors: Shuza Binzaid, San Antonio, TX (US); Shaneel Shuza, San Antonio, TX (US)

(21) Appl. No.: 13/998,661

(22) Filed: Nov. 21, 2013

(51) Int. Cl. G01N 33/00

(52) U.S. Cl. CPC G01N 33/0098
USPC 702/19

(57) ABSTRACT

Health of plants depends on amount and quality of minerals in soil, sunlight, water moisture in soil etc. A novel technique is applied and results found to be very accurate. Months of various experimentations posed no harm to the sample plants of different species. An electronic system is successfully designed and tested to monitor the health status of plants that incorporating to plant's internal fluid's condition. Plants have their own characteristics that can indicate status of their health; herein, experiments also included samples of Epipremnum pinnatum cv. aureum (i.e. a commonly called ‘Money plant’), used by detecting the activities of its internal stem sap. The system's functional flow chart and results are explained here. Extensive study is completed to determine a relationship of signals from a plant about its health status and translate it into a heartbeat-like signal, called as “Healthbeat”. A relationship of temperature and relative moisture of soil is established. Also table of collected data of relative soil moisture, daylight conditions and plant's health-beat during a day is presented here. This healthbeat module can easily be used with any other kind of plants and thus develop data base for each species of plants based on environmental and soil conditions by applying this technique.

### 1. BACKGROUND

### 1.1 Important Stem Tissues of Plants:

In vascular plants [Wunderlin, R. P., 2002], xylem is one of the two types of transport tissue, phloem being the other one (in FIG. 1). The xylem transports water from the root up the plant. The xylem is mainly responsible for the transportation of water and mineral nutrients throughout the plant. Xylem sap consists mainly of water and inorganic ions. Two phenomena cause xylem sap to flow, described below:

(i) Transpirational Pull: The most important cause of xylem sap flow is caused by the evaporation of water from the surface mesophyll cells to the atmosphere. Leaves play a major role in transpiration process. This transpiration causes millions of minute menisci to form in the cell wall of the mesophyll. The resulting surface tension causes a negative pressure in the xylem that pulls the water from the roots and the soil.

(ii) Root Pressure: If the water potential of the root cells is more negative than the soil, usually due to high concentrations of solute, water can move by osmosis into the root. This may cause a positive pressure that will force sap up the xylem towards the leaves. In extreme circumstances the sap will be forced from the leaf through a hydathode in a phenomenon known as guttation. Root pressure is most common in the morning before the stomata open and cause transpiration to begin. Different plant species can have different root pressures even in a similar environment.

Phloem is normally the inner tissue of stem and its main function is to transport sugars and other food materials from the leaves, where they are produced, to all other parts of the plant. This could be from the leaves to the roots to provide the chemicals needed for growth. However, it could be from a leaf and up to a developing fruit that is rich in sugars. The sugars are made by photosynthesis, which occurs in green parts of plants, such as leaves. The amino acids are made from sugars and minerals, such as nitrate absorbed from the soil. Phloem tissue is usually found close to the other transport tissue in plants, xylem, which transports water and minerals. In non-woody plants phloem and xylem are found in bundles, such as the veins of a leaf.

Phloem is composed of various specialized cells. It is composed of sieve elements and their associated companion cells, together with some sclerenchyma and parenchyma cell types. Sieve elements are long, thin-walled cells joined end to end, forming sieve tubes; large pores in the end walls allow the continuous passage of organic nutrients in particular, sucrose, a type of sugar that a plant need in all parts.

The transport tissues i.e. xylem and phloem, primarily flow vital liquids called sap that contains sugars, nitrates, water and ionized inorganic materials of chemical compounds. This ionic concentration is used to determine pH levels of sap. It is very important to know plants health by determining condition of pH and ionic contents of minerals.

### 1.2 Photosynthesis

Green plants are unable to live without sunlight. Photosynthesis is the process by which plants use the energy from sunlight to produce sugar, which is then converted into chemical forms of energy that can be used by biological systems. During this process, plants convert carbon dioxide (CO2) into organic material through the reduction of this gas into carbohydrates. The initial energy for this process is provided by the light of the sun, which is absorbed by pigments like chlorophylls and carotenoids. These chlorophylls absorb mostly blue and red light, while the carotenoids absorb blue-green light. Green and yellow light are not absorbed by the photosynthetic pigments in plants; therefore, these light colors are reflected by or passed through the leaves. This is why plants are usually green [Starr, F. and Martz, K., 1999].

Basically, Photosynthesis is the process when a plant turns the energy that it gets from the Sun into energy that the plant can use. When a plant has more sunlight it can photosynthesize faster because there is more sunlight for the plant to convert into energy. It is also important because it provides the energy that causes available water and carbon dioxide to react.

### 2. DESIGN OF HEALTHBEAT SYSTEM OF PLANTS

### 2.1 Materials and Methods

It is important to know soil forming contents, especially the concentration of soluble minerals which the plants' root can absorb. It is determined that the amount of these soluble minerals can be determined by measuring the ionic concentration in moisture or water in soil.

Electronic systems [Roden, M. S., 2002] today are used in many applications in biology. A new system is designed here to determine various vital information about the plant that are related to soil condition such as moisture, ionic concentration, sunlight intensity and plants transport tissue condition of sap. Four different types of sensors are used primarily to determine the relationship that is important to know about the plant.

Temperature Sensor: A very common sensor called ‘Thermistor’ is used in this project. Resistivity of sensor changes due to temperature is very linear; thus makes it a very simple attachment to the electronic circuits for the temperature sensing applications.

CdS Light Sensor: Light sensor of the system is very important for understanding the condition of active photosynthesis process in the leaves. During food processing, sucrose (glucose) and amino acid (protein) is produced in the green leaves of plants depending on the light intensity.

Soil Moisture Sensor: It is a very important part of the electronic system. Soil moisture and available ionic concentration depends on the amount of water in the soil. Soil's ionic concentration is determined a set of sensor probes in the soil. The electron permittivity is then measured to determine the available ions relative to soil moisture. Also longer period of electron injection can determine the steady-state soil conductance by changing ionic state of the minerals (in FIGS. 2, 3 and 4). This new type of sensor is made from chemically non-reactive ceramic material that is electrically extremely conductive. This small heat-tempered carbon-based ceramic sensor has two 2.5 mm×2.5 mm×25 mm probes 1.5 cm apart that are used to measure ion concentration at top soil for this particular experiment. Various dimensions of probes are required based on soil types.

Stem Sap Sensor: This special sensor is made of very hard carbon-based ceramic compound. It is also very sensitive to any small amount of ions in the sap that are touching on the surface of the sensor probes during sap flow in the stem of the plant. These probes are inserted in plant stem at least a distance of 10 cm from each other. So the differential state of ionic presence on probes due to the distance in stem can be used as a factor of ionic concentration of minerals in the plant's sap. This specially designed sensor is 0.6 mm×0.6 mm×7.5 mm long and used in the samples of Epipremnum pinnatum, i.e. Money plants. The sensor can also be designed based on the species of plant, and also its stem type and area.

Processing Unit: This is a small electronic processing unit [Sterpone, L., 2008] consisting of two IC (integrated circuits) chips. This circuit receives the analogous signals from sensors through copper wires that are specially insulated with micro-thin enamel. Wires are connected to ceramic sensor probes by application of special conductive gel compound. This unit produces combined information which later can be used for the plant's vital sign. Latter the combined information is used for producing audible beats, ion charge density and electron injection factors for conversion to conductance.

Health-beat Generator: A small electronic module that is attached to the sensor data processing unit. This module transforms the processed information into a relative frequency that is converted into heartbeat like sound by a tiny speaker. So the sound can be fast or slow paced based on the processed information as the plant's vital sign after system tuner adjustments.

### 2.2 Test and Discussion on Results

Electron injection is done by applying a low DC potential at ultra-low current levels. Injection process was done using a large capacitor 4700 uf to verify charge collection process based on time (in FIG. 3). In this test, capacitor acts as charge collector similar to soil as it has both positive and negative ions of solutes. This timing was measured for charges between ⅓ and ⅔ of the applied for collected charge potential to be in linear region of the capacitor. After determining the effective tuning parameters and safe levels of ultra-low current (ionic transfer) setup was completed that is controlled by a signal processing amplifier IC circuit, attached to soil moisture sensor (in FIG. 6). Data was collected repeatedly to confirm soluble ion contents in the soil, by giving 1 hour between set of data collection process [Binzaid, S. and Shuza S., 2012].

The second set of circuit, built with another IC, was connected to the sensors placed 10 cm apart (in FIG. 5) in the plant's stem. This makes a signal for differential change in plant's sap condition and also adjustment that the plant makes due to the soil moisture and its ionic variation incorporated with the sap flow and charges sensed in the sap. Data was collected to correlate and find the factor of effective tuning adjustments to electronic circuit for functional optimization for Epipremnum pinnatum plant. Such adjustments can easily be made for any other kind of plants using various carbon-based sensors also.

Light condition was measured by a simple CdS photo sensor connected to the system by the tuned signal amplifier. Similarly, a common thermistor sensor was incorporated with an amplifier for the system to monitor temperature, a factor for soil ion concentration and transpiration of plants.

After collecting all sensors data, final tuning adjustments were made to all system circuits as necessary. These adjustments were completed after correlating with pH, light and moisture meters bought from a local plant nursery. Thus the system was made ready to run the experiments for the plant after confirming its operational flow (in FIG. 8) of the circuit.

Plant's status was determined in few simultaneous data collection steps after watering the plant. These data were later plotted to understand the state and process of transpiration of plant related to the soil moisture level (in FIG. 9) and temperature. In changing moisture level of soil, it is very important to determine conductance of the soil at relative temperatures by plotting the data (in FIG. 10).

All data external to the plant were conductance, moisture and temperature; but they were very important information for determining the plant's sap condition, additional to the information that were collected by the sensor probes placed in the stem. Processing of the system is completed using the information generated by its respective amplifier circuits. An output signal generated by the system is then processed for an audible sound similar to an animal heartbeat. The system can generate a variable beat made by the processing status of the sap condition. As plants do not have heart, but their sap condition determines their living condition or vital status; the reason why this heartbeat like sound is called as ‘health-beat’ in this work. FIG. 11 shows soil pH, soil moisture and sap concentration from sensors signal (before amplifier) and referencing the time (i.e. 1/healthbeat) shown (a) morning, (b) afternoon and (c) night time. A plot is generated to show the health-beat (in FIG. 12) relative to the soil conductance which is also a complex function of soil's relative moisture, concentration of soluble mineral ions and temperature. A summarized test data is presented in Table 1.

### 3. TYPICAL EXPRESSION FOR SYSTEM ELECTRONIC PROCESS

A typical equation is expressed by,

$$F ( t ) = S ( f ) Z ( t ) d t = {\sum }_{k = 1}^{n} Y ( k ) {\sum }_{s = 1}^{m} \{ M ( s ) φ ( s ) \} d T$$

### Here,

F(t)=Healthbeat signal bit-rate

S(f)=Continuous signal at output

f=Averaging process of signal peak

φ(s)=Sensor's electronic specification value at linear region

k=Independent variables i.e. Soil, Sap, Stem growth etc.

s=Dependent variables of sensors i.e. moisture, solute ion, sap ion and flow, temperature, light

M(s)=Sensor pre-amp tuning factor

Y(k)=Factorizing-combinational value

Z(t)=Healthbeat signal timing factor

dT/dt=Change in temperature at any given time as required on daily, seasonal or annual basis

Note: parametric values and their characteristics can change based on the requirements of the system; thus the equation be altered.

### 4. FEATURES OF PLANTS HEALTHBEAT SYSTEM

This section lists the main features of the system. Other features are described in the subsequent sections. These features are:
Design is very flexible as various types of comparators and operational amplifiers can be used for the system.
Low power ICs can run the system at lower voltage and power.
System can be used to determine soil condition by monitoring ionic concentration of solutes and thus can indicate initial need for fertilizers for a particular plant under its specific environment.
System can be used for plants at home, garden and fields.

### 5. BRIEF DESCRIPTION OF THE DRAWINGS AND FIGURES

FIG. 1. Xylem and phloem tissues are like vein in stem of the plant (courtesy online photo source). They ransport nutritional fluids to every part of the plant.

FIG. 2. Heat-tempered carbon- based ceramic soil sensor. These sensor probes' dimensions vary based on the plants' stem and soil types.

FIG. 3. Ionic (charge) injection is being tested with a simple circuit supplying a digital pulse to a capacitor (showing in right hand)

FIG. 4. Initial setup of soil conductance test for Conductance=1/Resistance, and here showing Resistance=100.7 KΩ (KOhms)

FIG. 5. Two sensors' probe-pairs showed (1) placed 10 cm apart in the stem for sap ion and flow detection and (2) another in the soil for ionic contents in moisture.

FIG. 6. A typical sensor signal amplifier circuit. The design changes are made based on sensing requirements and environmental variables.

FIG. 7. A typical circuit of single digital output signal produced using the combinational input signals. Ultra-low power circuits are designed based on sensing requirements and environmental variables.

FIG. 8. Typical flow diagram of processing of sensors' information by the system. FIG. 9. Soil temperature and relative moisture in 24 hours. This is measure after soil is watered in the morning. FIG. 10. Soil temperature and conductance in 24 hours determined by ultra-low level electron injection process.

FIG. 11. Combinational Peak-signals of three sensors (before amplifier) at referencing time (second, S) and here Period of the Signal (s)=(1/Healthbeat); that is showing here during (a) morning, (b) afternoon and (c) night

FIG. 12. Relative soil conductance at ultra-low level electron injection and plant's healthbeat/min.

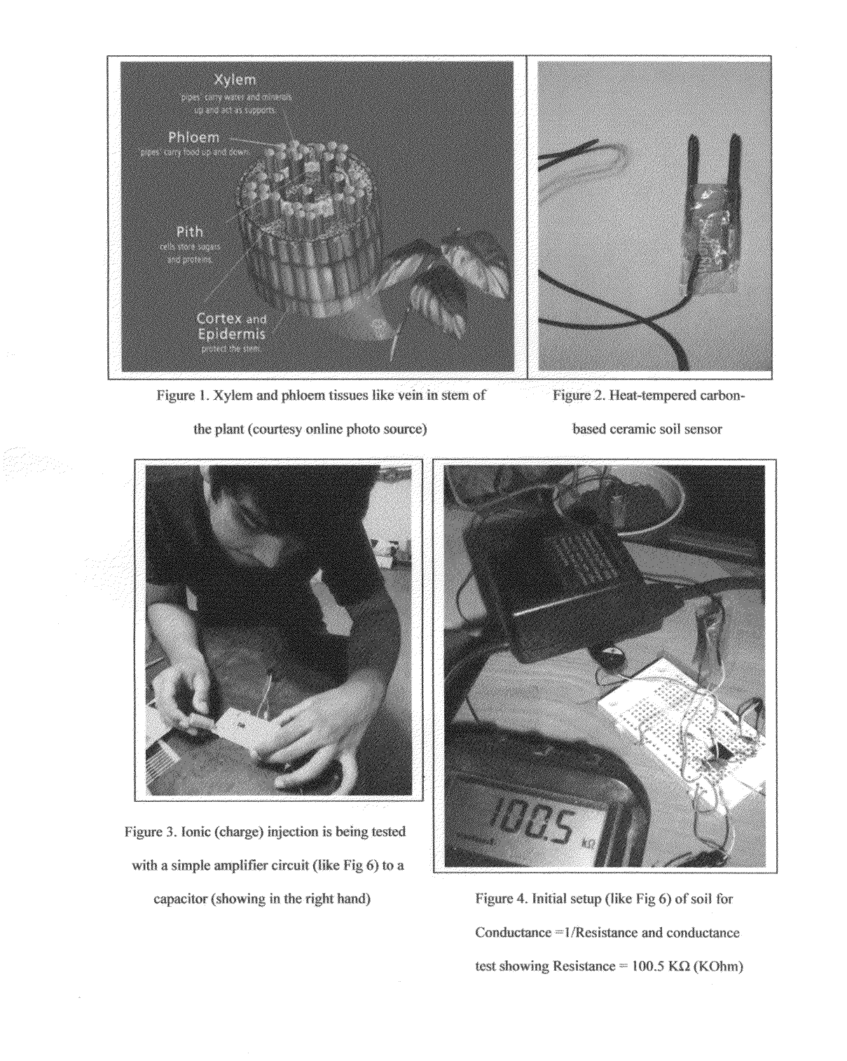

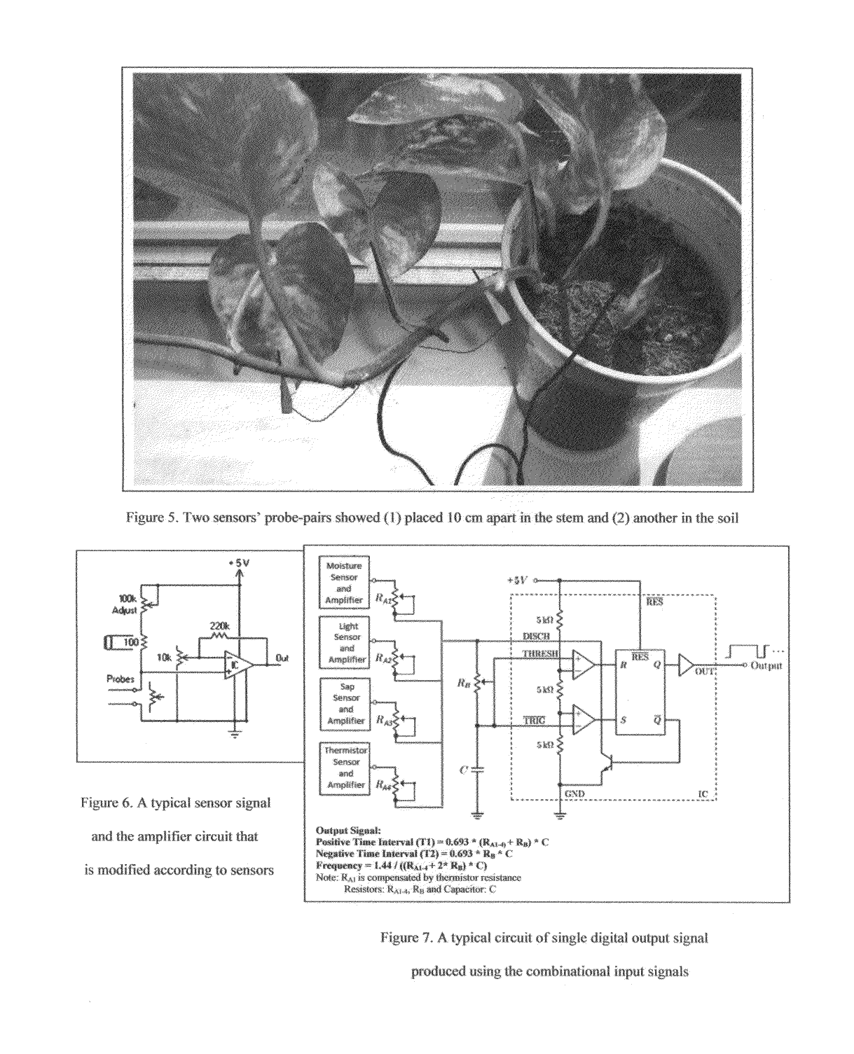

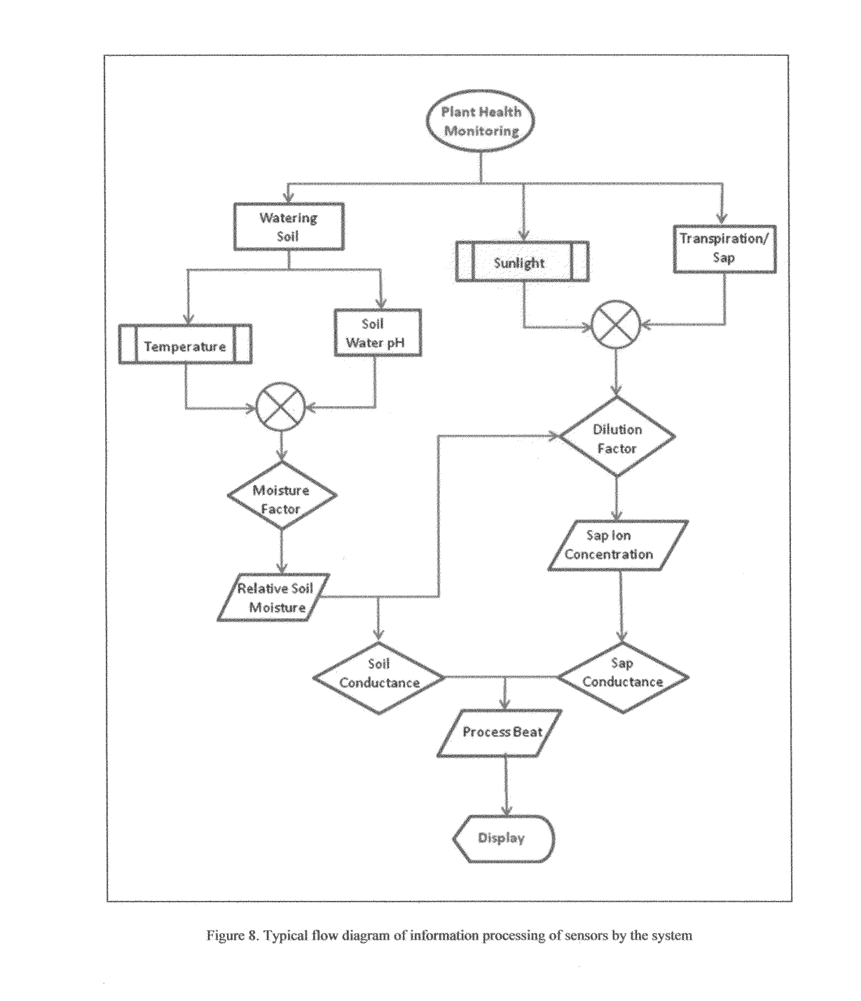

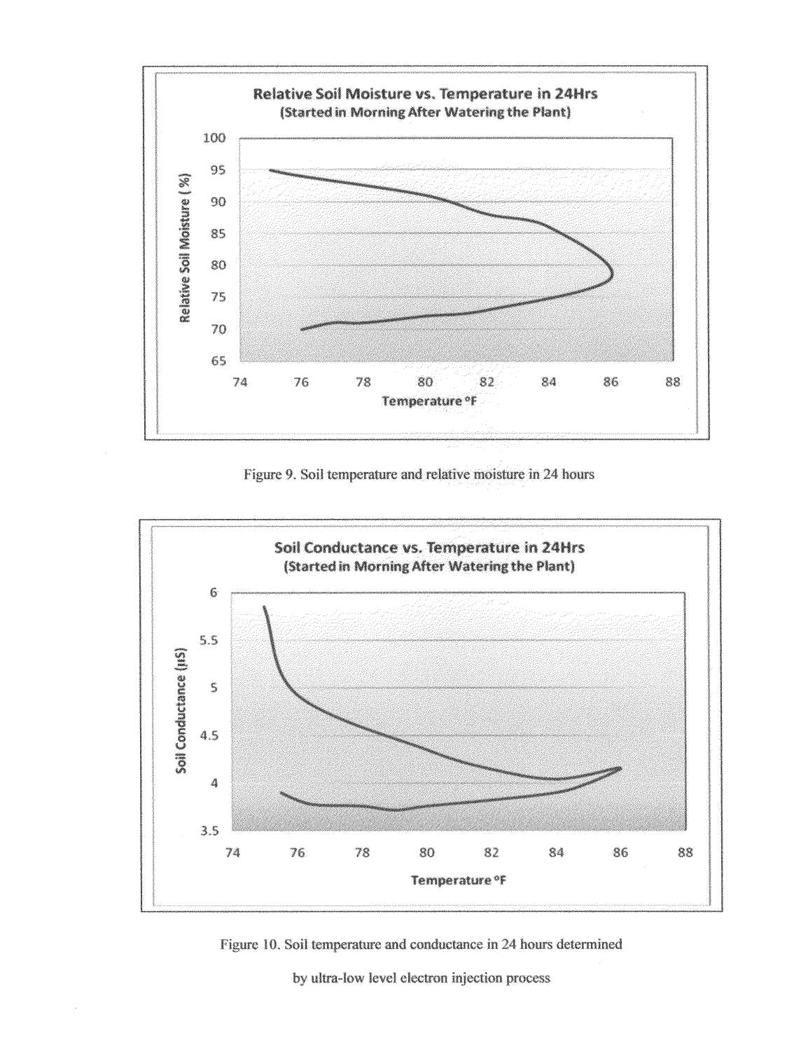

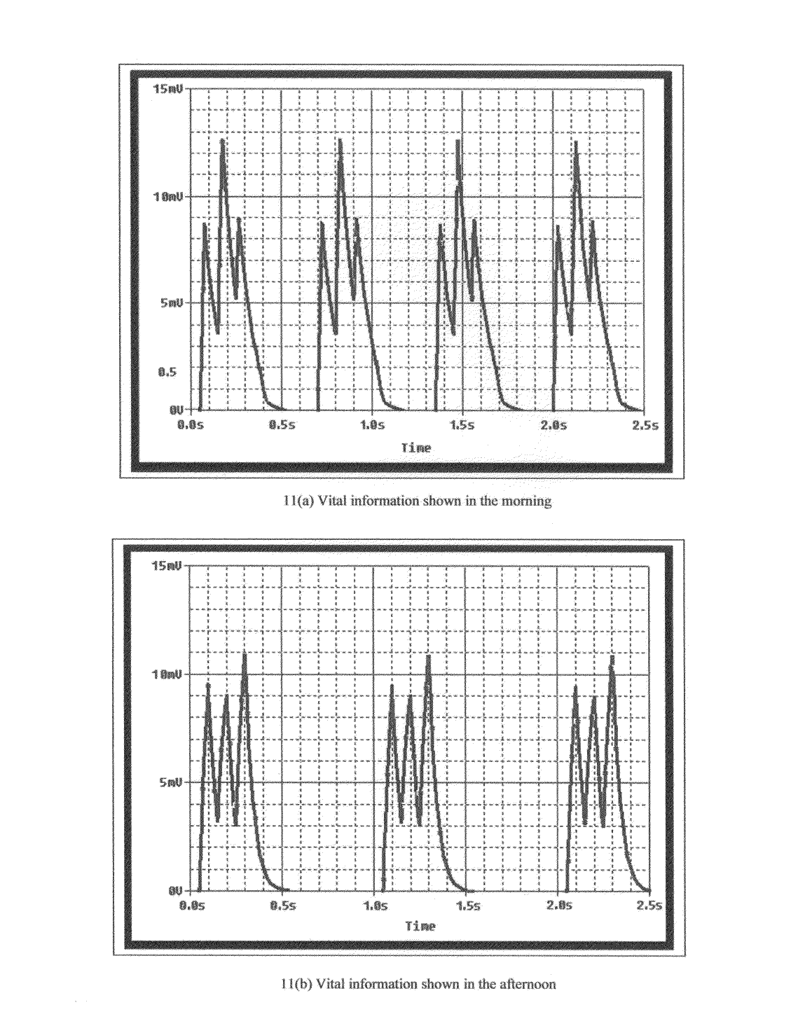

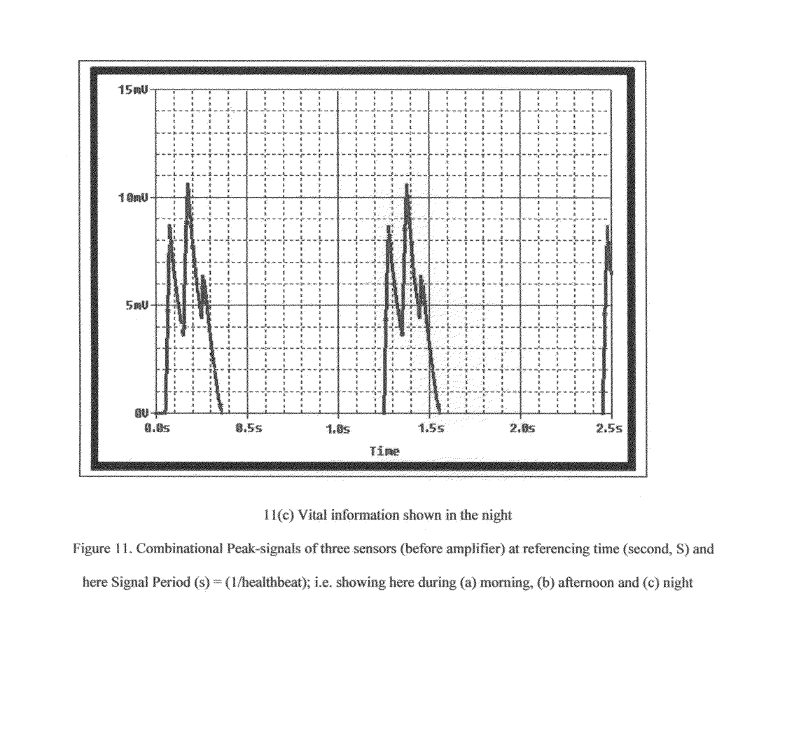

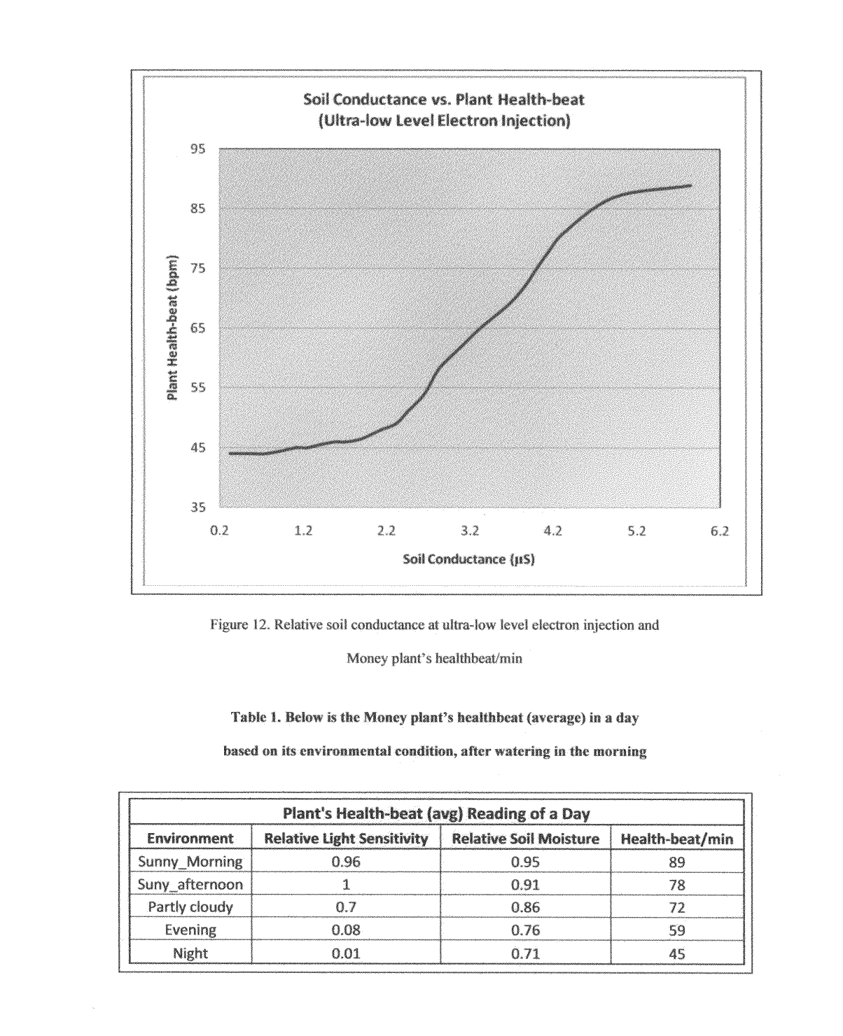

### 6. THE FIRST TEST AND THE FIRST SUCCESSFUL TEST OF THE INVENTION

### 6.1 The First Test:

A sensing amplifier was designed for a carbon-based ceramic sensor consisting 3 probes having dimension of 4 mm×4 mm×60 mm were used in the first successful test of plant sap information of a young Live Oak tree i.e. Quercus virginiana, having 12 ft height in September 2011.

### 6.2 Intermediate Tests:

Various experiments continued including vegetables and Rose plants using various dimensions of probes and amplifier circuits.

### 6.3 The First Successful Test of Invention:

During the months between May and September 2012, the best successful designs of amplifiers were tuned to factorizing-combinational circuit input setup. In October, the first successful system was used to collect data and verified the healthbeat of home grown common Money plant i.e. Epipremnum pinnatum cv. Aureum.

1. The system consists of low-power electronic circuits specially designed to monitor plants' vital status using various special process of factorizing-combinational information that are sensed by sensors including soil moisture and solute ions, temperature, light, plant sap conditions etc.

2. Claimed in 1, carbon-based ceramic material of non-corrosive, non-reactive and electrically conductive sensors that applicable to all ultra-low current sensing purposes by the healthbeat system regarding plant's sap and soil moisture contents.

3. Claimed in 1 and 2, the system is very adaptive to many sensors with variable dimensions and sensitivity of probes and also placing them at adjustable distances.

4. Claimed in 1, the system processes a simple electrical mixed-signal (analog and/or digital) output using factored-summing amplification of inputs.

5. Claimed in 3, adjustments of sensitivity are made by on-board tuner-component circuits of the system before measuring and monitoring health status of plants.

6. Claimed in 1, relationships between important variables are established where flow-chart shows the typical process of the system.

7. Claimed in 1 and 4, found mathematical expressions for the typical design of system to determine the appropriate health information.

8. Claimed in 4, an audible signal is formed using system's output signal that sounds like heartbeat, called ‘healthbeat’ for plants (as plants do not have any muscular heart).

9. Claimed in 4, visible signals are formed using system's output signal that (i) light-blinking at the rate of healthbeat and/or (2) a digital counter that shows the numerical value display.

10. Claimed in 1, the electronic healthbeat system, for monitoring plants vital conditions, can run with AC (includes AC-DC adapter), DC power (includes battery) and renewable (includes solar) energy sources of various voltages (a typical system is tested as low as 2.6V).
